(19) United States
(12) Patent Application Publication
(10) Pub. No.: US 2014/0322170 A1
(43) Pub. Date: Oct. 30, 2014

(54) Fermented Flavoring System Derived from Greek Yogurt Processing

(71) Applicant: GLANBIA NUTRITIONALS (IRELAND) PLC, Kilkenny (IE)

(72) Inventors: Earl Christiansen, Twin Falls, ID (US); Loren S. Ward, Twin Falls, ID (US)

(73) Assignee: Glanbia Nutritionals (Ireland) PLC, Kilkenny (IE)

(21) Appl. No.: 13/873,100

(22) Filed: Apr. 29, 2013

(51) Int. Cl. A23L 1/22; A61K 35/74

(52) U.S. Cl. CPC A23L 1/22091; A61K 35/741
USPC 424/93.4; 426/583

(57) ABSTRACT

Disclosed are methods for producing flavoring ingredients, ready-to-drink formulations, protein powder mixes and pre-mixes, yogurt-flavored cereal binders for use in cereal clusters and bars, etc., from Greek yogurt whey (i.e., Greek acid whey).

### RELATED APPLICATIONS

This application claims the benefit of priority of United States Provisional Patent Application No. 61/369,847, filed Apr. 27, 2012.

### FIELD OF THE INVENTION

The invention relates to compositions and methods for flavoring ready-to-drink formulations, powders, and other food products, and to compositions for use as ingredients in liquid and solid food products and nutritional supplements.

### BACKGROUND OF THE INVENTION

Milk and milk products comprise a significant part of the diet for many people around the world. Products derived from milk are as diverse as the cultures from which they originate. Grocery stores stock whole milk, reduced-fat milk, skim milk, evaporated milk, powdered milk, condensed milk, various cheeses, cream, butter, yogurt, and other products which have varied consistencies and flavors, making them suitable for a variety of uses. In various cultures, specialty products are popular. For example, dulce de leche, made by slowly simmering milk and sugar, is used in candies, cookies, ice cream, and other sweets in Central and South America. Cràeme fraiche is a higher-fat soured cream product that originated in France and is popular in many parts of Europe.

In many countries, fermented milk products are very popular for their tart taste and nutritional benefits. In India and Pakistan, for example, lassi is made by blending yogurt and water with spices, or sugar and fruit. Doogh, a yogurt-based beverage which may be carbonated, is popular in the Middle East. Yakult, which originated in Japan but is currently being produced and sold in several countries, is a Japanese product made by fermenting a mixture of skimmed milk (e.g., skim milk powder and sugar) with Lactobacillus casei Shirota. Another fermented beverage from Japan, Calpis, is a non-carbonated beverage made from water, nonfat dry milk and lactic acid. Methods for making fermented milk drinks have been the subject of patents such as U.S. Pat. No. 4,260,636 (Yasurnatsu, et al.)

Many other products similar to Yakult and Calpis are referred to as “drinking yogurt” and are made of stirred yogurt with a lower total solids content to provide a liquid or pourable consistency. Generally, it has also been homogenized to further reduce the viscosity. Yogurt is manufactured by fermenting milk with lactic acid bacteria. Fermented milk, such as Kefir containing 1 to 3% alcohol is made by adding yeast.

Fermented milk products such as Yakult and Calpis have been gaining in popularity and their markets have been expanding. Another product that has experienced a tremendous surge in popularity and market share is Greek yogurt, which is a variety produced by straining off the yogurt whey and thereby concentrating the remaining desirable components, most importantly the protein. According to the Los Angeles Times online (http://articles.latimes.com/2012/feb/24/business/la-fi-greek-yogurt-20120225), “Greek yogurt now hauls in more than $1 billion in revenue a year in the U.S.—about a quarter of total yogurt sales.” That article also indicates that sales of Greek yogurt have increased by 100 percent over the last three years. Production of Greek yogurt is in the hundreds of thousands of tons, and it takes about 4 gallons of milk to product one gallon of Greek yogurt. As a rough mass balance that means 1 gallon of yogurt and 3 gallons of Greek yogurt whey. This volume of production and the fact that the yogurt is produced by separating the whey and disposing of it have led to problems for the Greek yogurt industry, however, because the Greek yogurt whey has become a by-product for which Greek yogurt producers must pay others for its disposal. For example, in the Oneonta, New York community in which the Chobani plant is located, Chobani has been reported to “[pay] several local farmers to accept tank loads of it. The farmers said they use it as a fertilizer.” (http://thedailystar.com/localnews/x598832445/Officials-plan-meeting-with-Agro-Farma/print). Current pricing for that area is reported to be about five cents per gallon, with one local farmer being paid $300 per load of 6,000 gallons of Greek yogurt whey, which he mixes with manure and spreads on his fields. Although this is somewhat of a boon for the individual farmer and the local farming community, it represents a cost to the Greek yogurt manufacturer and it has caused some concern over potential environmental issues (http://thedailystar.com/localnews/x1125105293/State-beg ins-probe-of-Unadilla-River-mussel-kill/print).

Individuals who produce small quantities of Greek yogurt at home have indicated that the whey strained from their Greek yogurt may be used for bread-making and other uses where buttermilk may have previously been used. Some also report that Greek yogurt whey makes a good starter material for mixing with shredded cabbage to make homemade sauerkraut. Such uses, however, would not effectively account for the volume of Greek yogurt whey already produced each year, and the market and production are growing at a rapid rate. It would therefore be of great benefit to find commercial uses for Greek yogurt whey.

### SUMMARY OF THE INVENTION

The invention relates to a method for producing a ready-to-drink beverage, the method comprising adding at least one protein, in the absence of added yogurt, to a composition selected from the group consisting of Greek yogurt whey (GYW), concentrated Greek yogurt whey, dried Greek yogurt, and combinations thereof, to produce a protein-fortified Greek yogurt whey beverage. Aspects of the invention also provide protein-fortified Greek yogurt whey that has been processed by packaging the GYW into individual, single-use containers such as cans, glass bottles, plastic bottles, or drink boxes. In various aspects the method comprises the additional step of adding to the Greek yogurt whey additives selected from the group consisting of flavorings, stabilizers, juices, protein(s), vitamins, minerals, carbohydrates, prebiotic compositions, probiotic microorganisms, and combinations thereof. In various aspects, the at least one protein may comprise, for example, whey protein concentrate, whey protein isolate, milk protein concentrate, whey protein concentrate, soy protein isolate, soy protein concentrate, vegetable proteins, oil seed proteins or other edible proteins.

The invention also relates to a fermented flavoring system comprising base flavoring compositions selected from the group consisting of liquid Greek yogurt whey, concentrated liquid Greek yogurt whey (i.e, Greek yogurt whey concentrate), powdered Greek yogurt whey (i.e., dried Greek yogurt whey), and combinations thereof, the flavoring system effective for use in products selected from the group consisting of at least one food, drink product, confection, pharmaceutical product, and combinations thereof. These compositions impart a refreshingly tart flavor to the products.

In various aspects, the flavoring system further comprises compositions selected from the group consisting of flavorings, stabilizers, juices, protein(s), vitamins, minerals, carbohydrates, prebiotic compositions, probiotic microorganisms, and combinations thereof. The flavoring system may comprise, for example, powdered GYW, to which one or more additives selected from the group consisting of flavorings, stabilizers, juices, protein(s), vitamins, minerals, carbohydrates, prebiotic compositions, probiotic microorganisms, and combinations thereof have been added prior to, during or after the process of drying the powdered GYW. In other aspects, one or more additives selected from the group consisting of flavorings, stabilizers, juices, protein(s), vitamins, minerals, carbohydrates, prebiotic compositions, probiotic microorganisms, and combinations thereof may be added to powdered GYW after the drying process is completed.

The invention also relates to a method for preparing flavored protein mixes and/or pre-mixes, the method comprising admixing into to liquid Greek yogurt whey at least one protein to provide a protein/Greek yogurt whey mixture, and drying the mixture to form a protein/Greek yogurt whey powder.

### BRIEF DESCRIPTION OF THE DRAWINGS

FIG. 1 is a flow chart of processing steps that may be used to produce beverages, coatings, cereals, nutritional bars, flavored protein powders, and other products according to the methods of the invention.

FIG. 2 is a flow chart of processing steps describing the production of flavored mixes or pre-mixes such as, for example, a flavored milk protein concentrate.

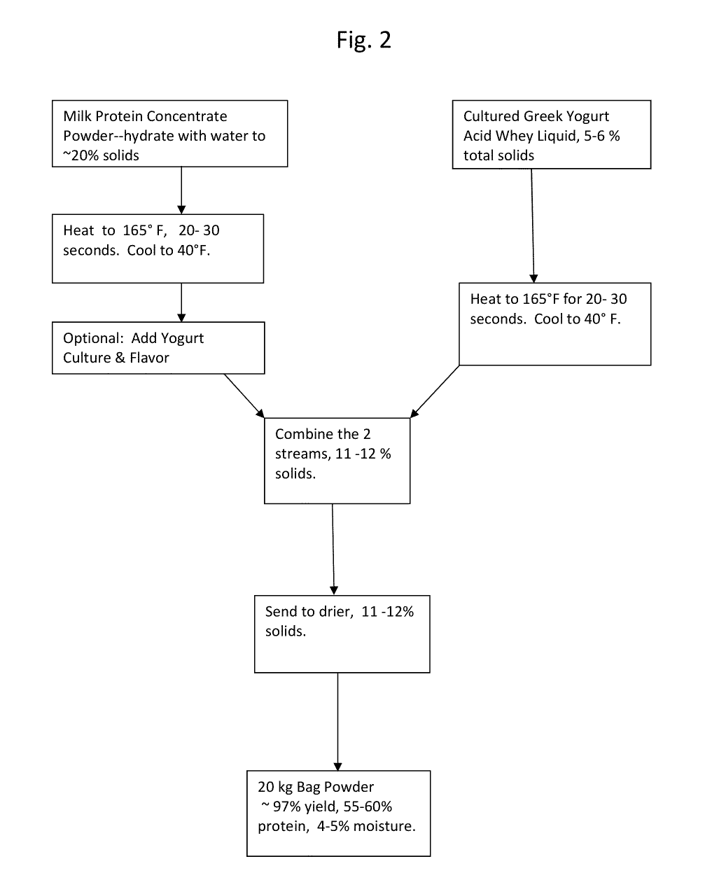

### DETAILED DESCRIPTION

The inventors have discovered that Greek yogurt whey (GYW), rather than being a by-product that creates disposal problems for the Greek yogurt industry, provides an excellent ready-to-drink product, a base for flavored ready-to-drink products, and the basis for a flavoring system for food products, drink products, medicinal products, etc., comprising liquid Greek yogurt whey, concentrated liquid Greek yogurt whey, and/or powdered Greek yogurt whey. The inventors have discovered that GYW provides an excellent flavor base for making ready-to-drink products, drink mixes, flavored concentrates, flavored syrups, pastes, powders, and other products, including those which can themselves be used as a flavor base for food and drink products, confections, and/or pharmaceutical/medicinal products. The inventors have also discovered that Greek yogurt whey, which is also known as Greek acid whey, can be used as an aqueous system to admix/dissolve other food components into, spray-drying the resulting mixture/solution, the method being effective for producing a wide variety of dried ingredients that can be used in other applications where a Greek yogurt flavoring system is desirable.

Fermented milk beverages are popular in many parts of the world, originating primarily in Asia and the Middle East, and their sales and popularity have spread to other geographic regions. Some of these beverages are made by purposefully combining milk, or milk powder and water, with bacteria or yeast, for example, to initiate the fermentation process and produce a drink with an acidic taste that many people consider to be tart, refreshing, and very pleasant. Other similar beverages are made by combining yogurt with water, optionally with other flavorings, sugar, etc., to produce a drink with a familiar yogurt taste. The production of Greek yogurt involves the removal of the components that many recognize as the milk/yogurt portion, leaving a type of whey that is somewhat unique to Greek yogurt processing. It is known as Greek yogurt whey (GYW). As used herein, “in the absence of added yogurt” refers to Greek yogurt whey (also known as Greek acid whey) to which no appreciable amount of yogurt has been added back after separation (e.g., for flavoring the Greek yogurt whey).

Table 1 lists the characteristics of the three main types of whey derived from the processing of dairy products. Whey may alternatively be referred to as “serum.” Some refer to Greek yogurt whey as “Greek yogurt serum” or “yogurt serum.” Sweet whey is manufactured during the making of cheeses like cheddar or Swiss (generally made using rennet, and known as “hard” cheeses). Unprocessed liquid cheese whey is regarded as nearly unpalatable in its original, unprocessed form. Acid whey (also known as “sour whey”) is derived from the process of making cheeses such as cottage cheese (generally small-curd) and is generally considered to be even less palatable than sweet whey. The beneficial aspect of acid whey and sweet whey is that the protein content of each is high enough and the lactose content is high enough that it makes industrial sense to isolate the protein and lactose and sell these ingredients into sports nutrition, infant formula or other food applications. Greek acid whey, or Greek yogurt whey, on the other hand, is not a product that the industry can utilize to economically recover protein. Furthermore, lactose in Greek yogurt whey is a challenge to crystallize due to the high lactic acid content of Greek yogurt whey.

TABLE 1

| | #01112, | #01114, | Greek Yogurt |
|---|---|---|---|
| | Acid | Sweet | Whey (Sample |
| | Whey* | Whey* | RD 12-178)** |
| Water (grams) | 93.42 | 93.17 | 94.51 |
| Calories (kcal) | 24 | 27 | 21 |
| Protein (grams) | 0.76 | 0.85 | 0.34 |
| Total lipid (fat, in grams) | 0.09 | 0.36 | 0.026 |
| Carbohydrate, by difference | 5.12 | 5.14 | 4.77 |
| (grams) | | | |
| Fiber, total dietary (grams) | 0 | 0 | 0 |
| Sugars, total (grams) | 5.12 | 5.14 | 4.77 |
| Minerals: | | | |
| Calcium, Ca (mg) | 103 | 47 | 113.5 |
| Iron, Fe (mg) | 0.08 | 0.06 | Not available |
| Magnesium, Mg (mg) | 10 | 8 | 10.9 |
| Phosphorus, P (mg) | 78 | 46 | 69.9 |
| Potassium, K (mg) | 143 | 161 | 143.9 |
| Sodium, Na (mg) | 48 | 54 | 41.3 |
| Zinc, Zn (mg) | 0.43 | 0.13 | Not available |
| Chloride (mg) | NA | NA | 130.95 |

*Source: USDA Nutrient Database
**Source: Glanbia Nutritionals internal analysis

Filtration membrane technology has progressed to provide various pore sizes and membranes that can fractionate or enrich a variety of components. Processing of Greek yogurt whey may include utilizing membrane technology for processing, such as nano-filtration. The molecular weight cut-off range of nano-filtration is from about 100 to 1000, with the membrane retaining whey solutes except monovalent charged ions. The resulting permeate consists of water, monovalent salts and some organic acids. Nano-filtration may be used to concentrate solids.

Reverse Osmosis (RO), for example, may also be used to concentrate solids. RO has a molecular weight cut-off range of less than 200. The membrane retains the whey solutes and the resulting permeate consists of water and some organic acids. Concentration of whey and UF permeate with Reverse Osmosis membranes can be achieved up to 20% maximum total solids.

Ultrafiltration is characterized as having a molecular weight cut-off range from about 3,000 to 100,000. The dairy standard is 10,000 MW, which is appropriate for fractionating whey proteins from lactose. This is commonly used to produce whey protein concentrates (WPC) of 35% to 85%.

The salt concentration of whey may be reduced by electrodialysis or ion exchange to produce demineralized whey. Lactose may be removed by enzymatic treatment with lactase, resulting in lactose-free whey, Whey may he dried using various methods, including the steps of preheating, concentration, flash cooling, pre-crystallization, spray-drying, and cooling. Equipment for producing powdered whey is available from sources such as, for example, GEA Process Engineering Inc., Columbia, Md., as are protocols for the use of such equipment to produce powdered whey. Greek acid whey may be concentrated through reverse osmosis and then a food component such as protein, fiber, or starch, for example, may be added to provide a concentration of from about 1 to about 95 percent. The resulting mixture may be co-dried, such as, for example, by spray-drying, to produce protein, fiber or starch-based ingredients with a refreshingly tart flavor similar to that of yogurt.

Ready-to-drink compositions and powdered drink mixes produced using a flavoring system of the invention may be used, for example, in hydration/recovery beverages. For example, a ready-to-drink composition comprising Greek yogurt whey without added yogurt may itself provide a hydration/recovery beverage because GYW contains a variety of minerals that are desirable for improving the body's electrolyte balance after exercise or dehydration. Alternatively, liquid Greek yogurt whey, concentrated liquid GM, powdered GYW, etc., optionally combined with other flavorings, stabilizers, juices, protein(s), vitamins, minerals, carbohydrates, prebiotic compositions, probiotic microorganisms, and combinations thereof, may be added to an existing hydration/recovery beverage. For example, U.S. Patent Application Publication Number 20110151059 describes a recovery beverage that comprises sodium, potassium, and other ingredients for rehydration and recovery, these being combined with suitable flavorings, which could comprise a flavoring system of the invention. Yogurt whey contains significant amounts of calcium, potassium, phosphorus and vitamin B12. Food and drink formulations made by using Greek yogurt whey therefore may have the added nutritional advantage of being a significant source of calcium, potassium, phosphorus and/or B-12 in the diet of individuals to whom the food or drink formulations might be provided. Various filtration and processing methods may, if desired, be used to enrich the various flavor fractions by removing residual lactose and minerals, providing multiple useful streams for ingredients tailored for specific flavor or nutritional applications. A “concentrate” of Greek yogurt whey may also be prepared using filtration and/or processing methods known to those of skill in the art for preparing concentrates from liquid compositions. Generally, such methods result in the removal of a portion of the liquid (e.g., water) fraction of a solution, suspension, etc.

Tart flavors such as that provided by fermented milk products such as yogurt are added to a variety of products as widely varied as yogurt-flavored pretzels, yogurt-flavored coatings on raisins and other fruit, and yogurt-flavored dog treats. Concentration liquid GYW and powdered GYW may provide a yogurt-like flavor to many products. For example, Quaker Yogurt Granola Bars (The Quaker Oats Company, Chicago, Ill.) provide a yogurt-flavored coating comprising sugar, palm kernel and palm oil, whey protein concentrate, yogurt flavored powder, soy lecithin, natural flavor, artificial color, citric acid, and salt. Greek yogurt whey powder, especially when combined with additional natural and/or artificial flavors, may provide an excellent yogurt flavor for such a product.

Improvements to the properties of cereal binders and reduction of the amount of sugar that must be incorporated into a cereal binder for producing agglomerated cereal products such as cereal clusters, bars, and other similar products, may be provided when at least one whey protein is incorporated into the binder. For example, compositions comprising binders for chewy cereal products, such as chewy cereal bars or clusters, including, for example, granola bars, breakfast bars, cereal bars, rice cakes, corn cakes, and popcorn cakes, may be formulated to comprise at least one sugar syrup and at least one milk protein, the protein comprising from about 0.2% to about 6% of the binder by weight. In various aspects, the protein may comprise from about 0.7 to about 6 percent of the binder, by weight. The method of the invention may be utilized to provide a flavored dry protein mix to produce a yogurt-flavored cereal binder comprising at least one sugar and at least one milk protein, admixed to form a binder for chewy clustered cereal products.

A binder for chewy clustered cereal products may also be produced by a method comprising the steps of hydrating whey protein at a level of about 25 to about 35 percent protein in water, and admixing the hydrated whey protein with at least one sugar syrup to produce a mixture having a viscosity solids range of from about 65 to about 85 percent, and even more preferably from about 70 to about 78 percent. The present invention may be utilized to provide yogurt-flavored whey protein for use in making such a binder for chewy clustered cereal products. Furthermore, the method may also involve utilizing Greek yogurt whey, or aqueous fractions thereof, to hydrate the whey protein prior to forming the sugar syrup.

Minerals derived from milk (“milk mineral,” generally comprising various minerals, but most particularly calcium and phosphorus, preferably in a ratio of about 2 to 1 calcium to phosphorus) have been utilized for preserving meat, for reducing the salt necessary to preserve meat, for maintain the color of meat products in their cooked and uncooked states, and for maintaining a desirable texture in cooked meat products. They have also been used as a mineral supplement, providing many of the essential minerals needed for human growth and development. Greek yogurt whey contains significant amounts of calcium and phosphorus, in a desirable ratio for purposes for which milk mineral has proven to be beneficial. Methods of the invention may also, therefore, comprise isolating milk mineral from Greek yogurt whey or concentrating the amount of milk mineral in a liquid or concentrate form of Greek yogurt whey to provide products for meat processing and nutritional applications. For example, desirable additives such as spices and flavorings may be added to liquid Greek yogurt whey, followed by the step of processing to concentrate the calcium and phosphorus content of the Greek yogurt whey into a liquid concentrate or a powder containing the desirable additives. Alternatively, spices and/or flavorings may be added to Greek yogurt whey which has already been processed to concentrate the calcium and phosphorus content of the Greek yogurt whey into a liquid concentrate or a powder. Greek yogurt whey also contains significant amounts of lactose, which may act as a tenderizer for meat. Compositions made as provided above may therefore be utilized as marinades, tenderizers, and/or preservatives that increase the desirable properties of meat during storage and/or processing (e.g., cooking). Compositions above may also be used in formulations to provide essential minerals and to provide an economical way to fortify food products such as bars, beverage and cereals with essential minerals.

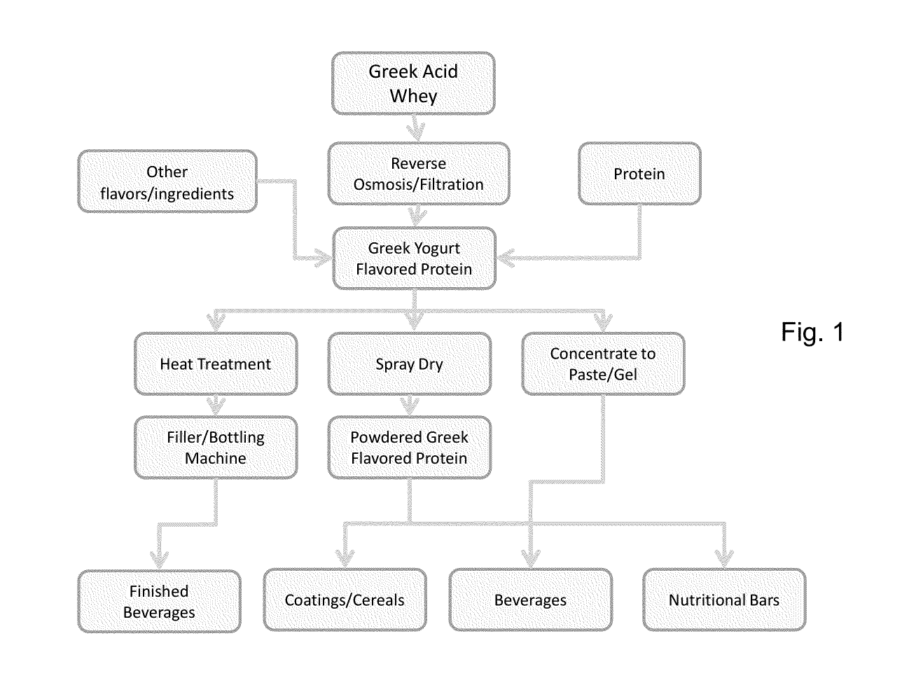

Bacterial fermentation produces desirable metabolites that can have a positive impact on health and wellness. Many metabolites are water soluble and are found in whey. Greek acid whey may provide additional nutritional benefits beyond milk minerals and be used as a source of bioactive metabolites for health purposes. FIG. 1 shows a diagram of how to process Greek whey and how it may be used in various food applications.

The invention may be further described by means of the following non-limiting examples.

### EXAMPLES

Making Ready-To-Drink Products from Greek Yogurt Whey

Shelf-stable ready-to-drink products were made by dry-blending milk protein concentrate, sugar, pectin and flavor together. Using a high shear mixer, the dry blend was slowly added to the GYW and mixed until homogeneous. Antifoam was added and pH was adjusted with phosphoric acid. The target pH of finished drink was 4.10. The shelf-stable product was thermally processed using either indirect heat (plate and frame or tubular) or direct heat (steam injection), at a preheat temperature of 76° C. and final heat temperature of 104° C., with an 18 second hold. Homogenization was performed in two stages, with a pressure of 1500 psi in the first stage and 500 psi in the second stage, at a temperature of 80° C. The ingredients used to make a pear-flavored drink product are listed in Table 2.

TABLE 2

| | |
|---|---|
| Greek Yogurt Whey | 87.714% |
| Milk Protein Concentrate | 0.975% |
| (Glanbia, Nutrasol 584) | |
| White Granulated Sugar | 10.0% |
| Pectin (Danisco, AMD 783) | 0.200% |
| Pear Flavor (Ungerer, Pear FT 7308) | 1.000% |
| Ginger (Ungerer, FT 7306) | 0.100% |
| Antifoam (Hydrite) | 0.001% |
| Phosphoric Acid | 0.010% |
| (Hydrite, 75% concentration) | |

The ingredients used to make a coconut-flavored drink are listed in Table 3.

TABLE 3

| | |
|---|---|
| Greek Yogurt Whey | 88.61% |
| Whey Protein Concentrate | 0.98% |
| (Glanbia, Avonlac 180) | |
| White Granulated Sugar | 10.00% |
| Pectin (Danisco, AMD 783) | 0.20% |
| Coconut flavor (OSF, 5871 A SD) | 0.20% |
| Antifoam (Hydrite) | 0.001% |
| Phosphoric Acid | 0.01% |
| (Hydrite, 75% concentration) | |

The ingredients used in an apple-flavored drink are listed in Table 4.

TABLE 4

| | |
|---|---|
| Greek Yogurt Whey | 88.464 |
| Milk Protein Concentrate | 0.975 |
| (Glanbia, Avonlac 180) | |
| White Granulated Sugar | 10.000 |
| Pectin (Danisco, AMD 783) | 0.200 |
| Apple Flavor (FONA, Nat 803.433) | 0.250 |
| Cinnamon Flavor (Ungerer, FU9699) | 0.100 |
| Antifoam (Hydrite) | 0.001 |
| Phosphoric Acid | 0.010 |
| (Hydrite, 75% concentration) | |

### Producing Flavored Dry Mixes

Greek acid whey was concentrated using reverse osmosis and protein was added to a concentration of 1-95%. The Greek acid whey/protein admixture was then spray-dried to produce a protein powder with Greek yogurt flavor which could be used in a variety of applications.

What is claimed is:

1. A method for producing a ready-to-drink beverage, the method comprising adding at least one protein, in the absence of added yogurt, to a composition selected from the group consisting of Greek yogurt whey, concentrated Greek yogurt whey, dried Greek yogurt, and combinations thereof, to produce a protein-fortified Greek yogurt whey beverage.

2. The method of claim 1 wherein the Greek yogurt whey is liquid Greek yogurt whey.

3. The method of claim 1 wherein the Greek yogurt whey is powdered Greek yogurt whey.

4. The method of claim 1 wherein the Greek yogurt whey is a liquid concentrate.

5. A composition selected from the group consisting of concentrated liquid Greek yogurt whey, powdered Greek yogurt whey, and combinations thereof, the composition being effective for use as a flavoring in at least one product selected from the group consisting of a food, drink product, confection, pharmaceutical product, and combinations thereof.

6. The flavoring system of claim 5 further comprising compositions selected from the group consisting of flavorings, stabilizers, juices, protein(s), vitamins, minerals, carbohydrates, prebiotic compositions, probiotic microorganisms, and combinations thereof.

7. A method for producing Greek yogurt flavoring ingredients, the method comprising admixing at least one ingredient into an aqueous Greek acid whey, in the absence of added yogurt, to produce an ingredient/Greek acid whey admixture, followed by a step chosen from among the group consisting of packaging the ingredient/Greek acid whey admixture, concentrating the ingredient/Greek acid whey admixture, and spray-drying the ingredient/Greek acid whey admixture.
